(19) United States
(12) Patent Application Publication
(10) Pub. No.: US 2023/0285668 A1
(43) Pub. Date: Sep. 14, 2023

(54) MEDICAL PRESSURE GAUGE

(71) Applicant: B. Braun Melsungen AG, Melsungen (DE)

(72) Inventor: Peter Mandry, Melsungen (DE)

(21) Appl. No.: 18/008,173

(22) PCT Filed: Jun. 2, 2021
(86) PCT No.: PCT/EP2021/064756

§ 371 Date: Dec. 4, 2022

(30) Foreign Application Priority Data
Jun. 5, 2020 (DE) 10 2020 207 084.9

(51) Int. Cl. A61M 5/168

(52) U.S. Cl. CPC A61M 5/16854; A61M 2205/75; A61M 2205/584

(57) ABSTRACT

A medical pressure indicator for infusion has a tube section that is combinable with a liquid-carrying filter element via an infusion line. The pressure indicator can expand and contract in at least one dimension depending on the pressure in the tube section.

A sterile packaged medical pressure indicator is connectable to the outlet of a medical infusion filter. The pressure indicator is at least partly made of elastic material that can expand and contract, and can be connected to the outlet of a medical infusion filter.

An infusion line can include the sterile packaged pressure indicator or an infusion filter with the medical pressure indicator.

### CROSS-REFERENCE TO RELATED APPLICATIONS

This application is the United States national stage entry of International Application No. PCT/EP2021/064756, filed Jun. 2, 2021, and claims priority to German Application No. 10 2020 207 084.9, filed Jun. 5, 2020. The contents of International Application No. PCT/EP2021/064756 and German Application No. 10 2020 207 084.9 are incorporated by reference herein in their entireties.

### FIELD

The present invention relates to a medical pressure indicator that can be coupled to a patient access port and that can easily and effectively detect an obstruction, blockage, or other flow disturbance in a medical infusion system using a specially formed pressure indicator. The present invention also relates to a sterile packaged pressure indicator for connection to the outlet of a medical infusion filter, and the use of said pressure indicator for connection to the outlet of a medical infusion filter. Finally, the present invention relates to an infusion line comprising the pressure indicator according to the invention, or an infusion filter comprising the pressure indicator according to the invention.

### BACKGROUND

In intensive medicine and intensive care, it is not only a question of the optimal therapy of the various clinical pictures, but always also a question of the stability of the pharmaceuticals in solutions, the chemical-physical compatibility of administered infusions (compatibility) and finally also a question of the compatibility of the pharmaceuticals with the disposable material (lines, cock banks, etc.). According to literature data, it is possible that in intensive care medicine about 10 million smallest particles find their way into the human organism within 24 hours per patient without an infusion filter. Infusion solutions can contain particles of different sizes, e.g. particles that are visible to the naked eye and have a size ≥50 μm, as well as particles that are not visible to the naked eye and have a size of less than 50 μm.

Glass ampoules, undissolved solids in drugs or parenteral solutions, or particle formation as a result of incompatibilities, for example between a drug and a carrier solution, represent a particularly high risk of particle contamination of infusion solutions. Particularly with the latter, so-called mixed infusions, the risk of particle contamination is especially high. Mixed infusions are prepared on an on-demand basis, for example in pharmacies or hospitals; the risk of particle contamination is particularly high in emergency situations.

The contaminating particles that are present in the intraluminal compartment and are not retained, for example, by a filter, enter the patient's body directly and can cause undesirable effects there. The risks of particle contamination include impaired microcirculation, displacement of blood vessels, damage to various organs, or phlebitis.

These complications can—in addition to harming the patient—also lead to additional treatment costs caused by a longer hospital stay.

To avoid such disadvantages, it is advised to use filter elements such as infusion filters. However, the use of infusion filters is not standard practice, as the correct handling of them must first be learned. In particular, nurses who are generally entrusted with administering infusions to patients suffer from this. It is therefore not uncommon for infusion filters, whose use would offer a not inconsiderable advantage, to be discontinued due to the initial handling difficulties, at least to the disadvantage of the patients, who are then exposed to an increased risk of contamination.

One of the problems that can arise in the handling of infusion filters is that if the infusion solution does not reach the patient as intended, it is not readily possible to identify the reason for this: It is not readily possible to distinguish whether the infusion filter is blocked (obstructed or blocked), or whether the patient access port is blocked (plugged or obstructed), for example, because the infusion needle is lodged against the vessel wall, or whether there is catheter occlusion. Because there has been no way to determine whether the infusion filter or the patient access port is plugged, it has been necessary to replace the infusion filter in the event of failure to pass infusion solution into the patient, without any certainty that this will solve the problem of infusion solution being delivered to the patient at the desired flow rate.

Due to this difficulty, among others, the use of an infusion filter is therefore often refrained from, although the use of such a filter is desirable and also recommended.

### SUMMARY

The present invention is now based on the objective of providing a medical pressure indicator which makes it comparatively easy to determine whether the infusion filter or the patient access port is blocked, and thus allows an infusion filter to be used more reliably than before.

According to the invention, the problem is solved by a medical pressure indicator for infusion, which is combinable, preferably combined, with a liquid-carrying filter element via an infusion line, wherein the pressure indicator is formed as a liquid-carrying element downstream of a combinable liquid-carrying filter element, wherein the pressure indicator can expand or contract in at least one dimension depending on the pressure in the liquid-carrying element. In a particularly advantageous embodiment, the liquid-carrying element—i.e., the pressure indicator—is a liquid-carrying tube section that can expand and contract in at least one dimension as a function of the pressure in the tube section. Such a pressure indicator, which can expand and contract in at least one dimension, is capable of indicating both overpressure (positive pressure) and underpressure (negative pressure) continuously, reliably and in a simple manner. Even very low pressures and pressure changes can be detected and measured. In addition, the pressure indicator has a simple design due to the smallest possible construction, which is adapted to the infusion tubing, and it allows, also in a simple way, to determine whether and where there is a blockage, at the infusion filter or at the patient access port. The above-mentioned advantages are also important when used on the patient, as the risk of the infusion getting stuck and thus being pulled out unintentionally is minimized. The configuration as a liquid-carrying tube section thus not only permits a small design, but also increases certainty due to the associated shape.

Further subject-matters of the invention are a sterile packaged pressure indicator suitable for connection to the outlet of a medical infusion filter, a pressure indicator combined with a liquid-carrying filter element, and the use of said pressure indicator for connection to the outlet of a medical infusion filter, and an infusion line comprising said pressure indicator or said infusion filter.

According to the invention, a medical infusion filter can be combined with a pressure indicator. The pressure indicator is formed—downstream of the filter element—as a liquid-carrying element. The pressure indicator has the property that it can expand or contract in at least one dimension depending on the pressure in the liquid-carrying element.

The term “downstream” refers to the general direction of flow of the infusion, that is, from the side of the infusion bag or infusion filter toward the side of the patient access port.

The term “pressure”, unless otherwise described, usually means the infusion pressure prevailing in the infusion system in which the filter element is located, in particular the pressure prevailing between the filter element and the patient access port.

Medical infusion filters can remove or retain particles from the infusion solution. As a result, contamination of patients with (micro-) particles to which an infusion solution is supplied can at least be reduced, and in the best case even avoided altogether. Such infusion filters are known to the skilled person and are available, for example, from the company B. Braun, Germany. Infusion filters are also described in the following publications: DE 3347374 A1, EP 2 567 720 A1, EP 0 784 988 A1.

The term “medical” as used herein means that the type of materials is suitable for passing an infusion solution, which means that no unacceptable/harmful substances are allowed to dissolve. For patient use, the medical infusion filter must also be sterile, so that the present invention preferably relates to a sterile packaged medical pressure indicator or a medical infusion filter with a pressure indicator.

The terms “expand” and “contract”, as used herein, each relate to a dimensional or size change of the pressure indicator to an extent that can be perceived by the naked eye or detected using a simple measuring device such as a scanner. In particular, such a dimensional or size change is perceptible to the eye or easily detectable by a suitable device in a pressure range of 1-70 mmHg. In particular, the term denotes a dimensional change in the millimeter range or even centimeter range, for example an expansion or contraction of at least 1 mm.

The pressure indicator may expand and contract in at least one dimension, for example radially to the direction of flow of the infusion solution.

The pressure indicator is located downstream after the filter element. During infusion administration, both the filter element, for example the filter membrane, and the patient access port can become blocked. There are basically three states in this system during infusion administration:
State 1: This state exists when all liquid-carrying parts are free from blockages or other disturbances of the liquid flow or pressure-changing situations. In this case, in the state connected to a patient, the pressure between the filter element (also referred to here simply as the “filter”; for example, the filter membrane of the infusion filter) and the patient access port corresponds to the static pressure due to the infusion solution minus the static venous pressure and the dynamic pressure of the infusion and blood flow. In this case, the pressure indicator is in a medium state or normal tension. This can be seen, for example, by the fact that a correspondingly formed line section between the filter element and the patient access port has a normal cross-sectional diameter or cross-sectional area. This means that although the pressure indicator does not indicate a change in dimension or size, it still has the ability or property to expand or contract depending on the change in pressure. In this state, i.e. when all liquid-carrying parts are free of blockages or other disturbances and the infusion solution can thus be supplied to the patient without hindrance, the pressure indicator is thus neither expanded nor contracted. This state is exemplified in FIG. 1A.

In this state (“normal state”), the diameter of the liquid-carrying element of the pressure indicator is at least 2 mm in the normal state.
State 2: This state exists if the patient access port is plugged or partially plugged. In the case of a complete blockage, the pressure between the filter element and the patient access port corresponds to the static pressure due to the infusion solution. The pressure indicator is at maximum tension. I.e. in this state the pressure indicator is maximally expanded. This state is exemplified in FIG. 1 B. This can be seen, for example, by the fact that the correspondingly formed, in particular elastic expandable line section between the filter element and the patient access port has a larger cross-sectional diameter or cross-sectional area than in the normal state (i.e., compared to the state shown in FIG. 1A).

In this state (“expanded state”), the diameter of the liquid-carrying element of the pressure indicator is at least 3 mm.
State 3: This state is present when the filter element (for example, the filter membrane of the infusion filter) is plugged or partially plugged and the infusion pressure applied upstream of the filter element no longer reaches the pressure indicator. In the case of a complete blockage, the pressure downstream of the filter element, i.e. between the filter element and the patient access port, corresponds to the venous pressure minus the dynamic pressure of the blood flow. The pressure indicator is relaxed. I.e. in this state the pressure indicator is contracted. This state is exemplified in FIG. 1 C. This can be seen, for example, by the fact that the correspondingly formed, in particular elastic expandable line section between the filter element and the patient access port has a smaller cross-sectional diameter or a smaller cross-sectional area than in the normal state (i.e. compared to the state shown in FIG. 1A).

In this state (“contracted state”), the diameter of the liquid-carrying element of the pressure indicator is at least 1 mm.

It is clear from the above explanation that the extent of the dimensional or size change that can be detected on the pressure indicator corresponds to the extent of the pressure change or the extent of the plugging and can lie between the extremes of “free” or “trouble-free” on the one hand and “completely plugged” on the other hand.

The state of the pressure indicator can therefore be used to quickly identify the cause and location of an interruption or malfunction in the fluid flow of the infusion solution. Depending on whether the pressure indicator is expanded (stretched) or contracted, it can be easily and reliably detected whether the patient access port or the filter element is at least partially plugged (blocked): If the filter element is at least partially plugged, the pressure indicator is—more or less—in the contracted state. If the patient access port is at least partially plugged, the pressure indicator is—more or less—in the expanded state. Depending on where the blockage is present, appropriate measures can then be taken to remove the blockage. For example, the at least partially plugged filter element can then be replaced. Since the medical infusion filter according to the invention comprising a pressure indicator can be used to detect comparatively quickly and reliably in a simple manner where the blockage is located (patient access port or filter element), and this can then also be removed, the present invention can lead to greater acceptance of the use of infusion filters. In addition, the solution proposed herein is comparatively inexpensive.

In one possible embodiment, the pressure indicator is contracted by at least 1 mm when the filter element is completely blocked at a standard infusion solution pressure upstream at the filter of 5 mmHg. In a further possible embodiment, the pressure indicator expands by at least 3 mm when completely blocked at the patient access port at a standard pressure of the infusion solution upstream at the filter of 70 mmHg (preferably referred to the non-expanded state and referred to normal pressure 1.013 bar and 20° C., for example a state in which the tubing is filled with air and the same pressure prevails inside and outside).

In a preferred embodiment, the pressure indicator is configured as a liquid-carrying element. Most expediently and most simply, this liquid-carrying element is a line section arranged downstream of the filter element, in particular a line section of an infusion tube or an infusion system between the filter and the patient access port.

In a preferred embodiment, the pressure indicator, or at least parts of the liquid-carrying element referred to above, is at least partially made of flexible material, such as elastic or non-elastic material, which is shaped to expand or contract. An example of flexible, non-elastic material is LDPE (“low density polyethylene”).

For example, the pressure indicator can be formed as an elastic tube section. A good and simple visibility of the pressure indicator is given if the tube section preferably expands from a pressure of >30 mmHg, preferably from greater than 40 mm Hg on the line and/or contracts from a pressure of <20 mmHg, preferably of <10 mmHg, whereas other parts of the tube do not expand at the indicated high pressure value or do not contract at the indicated low pressure value. This part of the hose serves as a reference to the dimensional change of the elastic part. A change can then be more easily detected by the observer. The tube section is made of elastic material, such as silicone, PVC (polyvinyl chloride), especially plasticizer-free PVC, or PVC containing the plasticizer DEHT (diethylhexyl phthalate). Preferred is PVC that contains DEHT as the only plasticizer.

In a further embodiment, it is also possible for the pressure indicator of the medical infusion filter to be formed as a line section with folds, wherein the pressure indicator can expand in the longitudinal direction from a folded normal state in the event of increased pressure and, if necessary, contract (more) from a folded normal state in the event of reduced pressure along this longitudinal direction. Such an embodiment is shown, for example, in FIG. 2.

In a further embodiment, it is possible and usable in a simple manner if the pressure indicator of the medical infusion filter is made partly of elastic material and partly of non-elastic, hard material. For example, the pressure indicator may be in the form of a membrane. The membrane may expand in a first direction due to the elastic material forming the membrane in response to increased pressure, representing an increase in pressure in the fluid system due to at least partial blockage at the patient access port. An example of this is shown in FIG. 3, which shows how the elastic membrane expands outward (“first direction”). Alternatively, due to the elastic material forming the membrane, the membrane may also expand in a second direction opposite to the first direction in response to reduced pressure, representing a pressure drop in the fluid system due to at least partial blockage of the filter. This is not explicitly shown in FIG. 3; however, it is readily apparent from this that the elastic membrane can expand inwardly (“second direction”).

The expansion and contraction of the pressure indicator, preferably the elastic part of the pressure indicator, preferably takes place radially to the flow direction of the infusion. However, it is also possible that the expansion and contraction takes place in the direction of flow of the infusion solution.

In one embodiment, the elastic material of the pressure indicator may be a membrane. This membrane may be opposed by a non-elastic (hard) component, such that the pressure indicator can only expand or contract on the side of the membrane. An example of such an embodiment is shown in FIG. 3.

The term “membrane” as used herein in connection with the elastic material of the pressure indicator does not refer to a semi-permeable membrane or filter membrane, but to a fluid impermeable thin “skin” of the line section or pressure indicator that can expand or contract.

In a further embodiment, or in addition to the embodiments described herein, it is possible that the filter is a capillary membrane.

In the case of a capillary membrane, the infusion solution is preferably filtered from the inside to the outside, wherein the outer skin of the line section surrounding the capillary membrane may comprise the elastic material of the pressure indicator, which may then expand or contract. Again, the elastic material is located “downstream” of the filter element because the filtered infusion solution that is in contact with the elastic material has already passed through the capillary membrane.

The medical infusion filter can be formed in one-piece, two-piece or multi-piece. In two-piece or multi-piece forms of construction, the infusion filter may be spatially separated from the pressure indicator, wherein both serve as a line section and are connected to each other, for example by an infusion tube.

Depending on whether the filter is fully or partially plugged, or whether the patient access port is fully or partially plugged, the pressure indicator expands or contracts to such an extent that the change in pressure indicator is visually detectable. This expansion or contraction of the pressure indicator occurs, for example, at a pressure in the range of 0-70 mmHg. Preferably, the pressure indicator, which may for example be formed as a tube section, expands from a pressure of greater than or equal to 30 mm Hg, preferably from greater than or equal to 40 mm Hg, and/or contracts from a pressure of <20 mm Hg, preferably of <10 mm Hg.

The pressure indicator according to the present invention is capable of continuously and reliably measuring negative and positive pressures, for example pressures as specified herein. It is thus also capable of measuring low pressures and thus indicating even comparatively small pressure changes.

In a preferred embodiment, it is only the elastic material of the pressure indicator that visually detectable expands or contracts at the certain pressure.

In order to make it easier to visually detect a change in the pressure indicator (i.e., an expansion or a contraction), it is preferred that a color marking is applied to the pressure indicator, the detectability of which changes with the expansion or contraction, preferably the expansion, of the pressure indicator, preferably the flexible (preferably the elastic) material of the pressure indicator.

The color marking of the pressure indicator of the medical infusion filter can create a different color impression depending on the state of expansion. This can further increase the visual perception of any change in the pressure indicator.

For example, the elastic material, such as a membrane, can be arranged over a color marking that becomes more visible when the membrane is stretched more.

The diameter of the pressure indicator of the medical infusion filter in the relaxed state is basically dependent on the shape of the pressure indicator. Thus, the pressure indicator may have any shape suitable for passing an infusion solution therethrough and indicating any pressure change that may occur by expansion or contraction in at least one dimension. For example, the pressure indicator may be tubular, accordion-shaped, round, or spherical.

For example, if the pressure indicator is tubular, the diameter of the pressure indicator in the relaxed state (liquid empty state at ambient pressure) is approximately equal to the diameter of the infusion tubing, for example, 0.7 times to 2 times the diameter of the infusion tubing. In a preferred embodiment, the diameter of the pressure indicator in the relaxed state is 2 mm to 8 mm.

The pressure indicator according to the present invention is thus of comparatively simple construction. The construction of the pressure indicator can thereby be adapted to the infusion line with which it can be combined, for example with respect to the size of the pressure indicator. This is an advantage, for example, in use on the patient: disadvantages in use can be avoided by keeping the size as small as possible, preferably in combination with a design adapted to the infusion tubing. For example, the pressure indicator can be prevented from getting stuck and pulling the infusion out of the patient as a result. Therefore, not only the simple design of the pressure indicator, which is preferably as small as possible, but also the shape of the pressure indicator being as simple as possible contribute to an advantageous use on the patient.

The present invention also relates to a sterile packaged pressure indicator for connection to the outlet of a medical infusion filter, wherein the pressure indicator is formed as a line section for the infusion solution, and wherein the pressure indicator is capable of expanding or contracting in at least one dimension depending on the infusion pressure in the line section. Advantageously, the sterile packaged pressure indicator is made at least in part of elastic material that can expand and contract.

All features as described herein for the pressure indicator in connection with the medical infusion filter also apply to the sterile packaged pressure indicator for connection to the outlet of a medical infusion filter.

The present invention also relates to the use of the sterile packaged pressure indicator described herein, for connection to the outlet of a medical infusion filter.

Finally, the present invention relates to an infusion line comprising a sterile packaged pressure indicator as described herein, or an infusion filter with pressure indicator as described herein.

### BRIEF DESCRIPTIONS OF THE DRAWING FIGURES

In the following, exemplary embodiments of the invention are explained in more detail with reference to the drawings; however, the drawings and the following description are intended to illustrate the invention in more detail without limiting the invention thereto.

FIG. 1 shows a pressure indicator according to a first embodiment.

FIG. 2 shows a pressure indicator according to a second embodiment.

FIG. 3 shows a pressure indicator according to a first embodiment.

### DETAILED DESCRIPTION

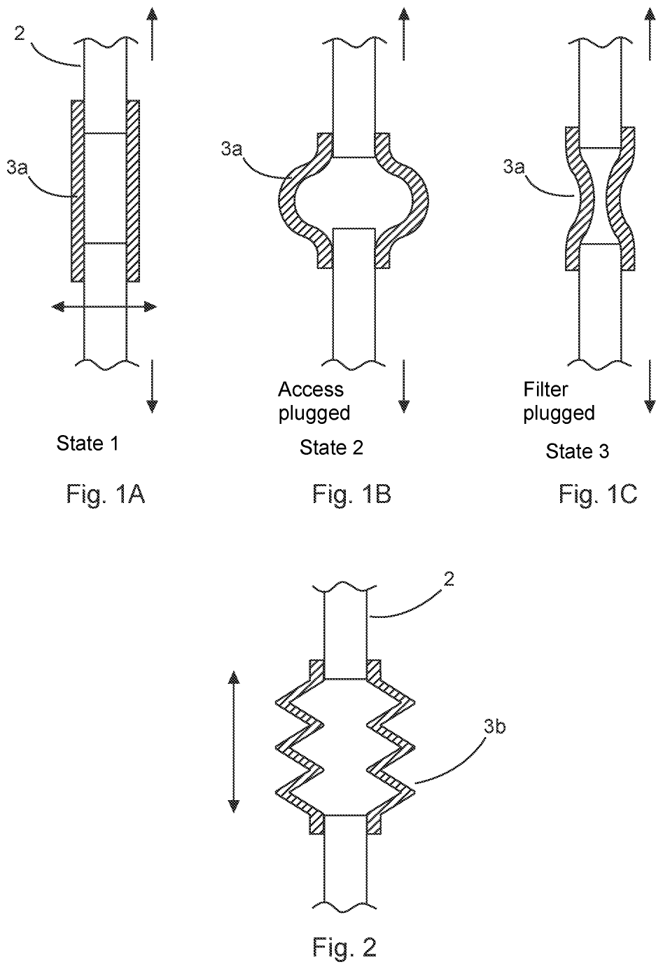

FIGS. 1, 2 and 3 each show examples of the first, second and third embodiments according to the invention. They each depict, in longitudinal section, a portion of a pressure indicator according to the invention coupled in fluid communication with a medical infusion filter. The filter element of the infusion filter itself as well as the patient access port are not explicitly shown; accordingly, any conventional or newly adapted filter elements to the pressure element or any conventional or newly adapted patient access forms to the pressure element can be used.

FIG. 1 shows the first embodiment, as well as the three possible states A), B) and C). In this figure, the infusion line 2 is shown downstream (arrow pointing up) of the filter element 1 (not shown) and leading to the patient access port 5 (not shown), arrow pointing down. A line section 3 of the infusion line 2 represents the pressure indicator 3. The pressure indicator 3 is here exemplarily formed as an elastic tube 3a.

FIG. 1A) shows state 1. This state 1 is present when the liquid-carrying parts (filter element 1, patient access port 5) are free. In this case, the pressure between the filter element and the patient access port 5 corresponds to the venous pressure plus the pressure drop across the infusion cannula and across the fluid path from the cannula to the filter element. In this case, the pressure indicator 3a is in an intermediate state or normal tension, which means that it can expand or contract, depending on the change in pressure. In this state, i.e. when the liquid-carrying parts (filter element 1, patient access port 5) are free and the infusion solution can thus be supplied to the patient unhindered, the pressure indicator 3a is thus neither expanded nor contracted.

In FIG. 1 B), there is a state (state 2) in which the patient access port 5 is at least partially blocked (plugged). In the case of a complete blockage, the pressure between the filter element 1 and the patient access port 5 corresponds to the static pressure due to the infusion solution. The pressure indicator 3a is maximally tensioned. I.e. in this state the pressure indicator 3a is maximally expanded.

FIG. 1 C) shows a state in which the filter element 1 (for example the filter membrane of the infusion filter) is plugged or partially plugged and the infusion pressure applied upstream to the filter element 1 no longer reaches the pressure indicator (state 3). In the case of complete blockage, the pressure downstream of filter element 1, that is, between filter element 1 and patient access port 5, is equal to the venous pressure minus the dynamic pressure of the blood flow. The pressure indicator 3a is relaxed. That is, in this state, the pressure indicator 3a is contracted. Since the venous pressure is very low, the pressure in the pressure indicator can reach negative values depending on the height at which it is attached.

FIG. 2 shows the second embodiment. In this embodiment, the infusion line 2 which is located downstream of the filter element 1 (not shown) and which leads to the patient access port 5 (not shown) can be seen. A line section 3 of the infusion line 2 represents the pressure indicator 3. The pressure indicator 3 is formed here exemplarily as an accordion-shaped pressure indicator 3b.

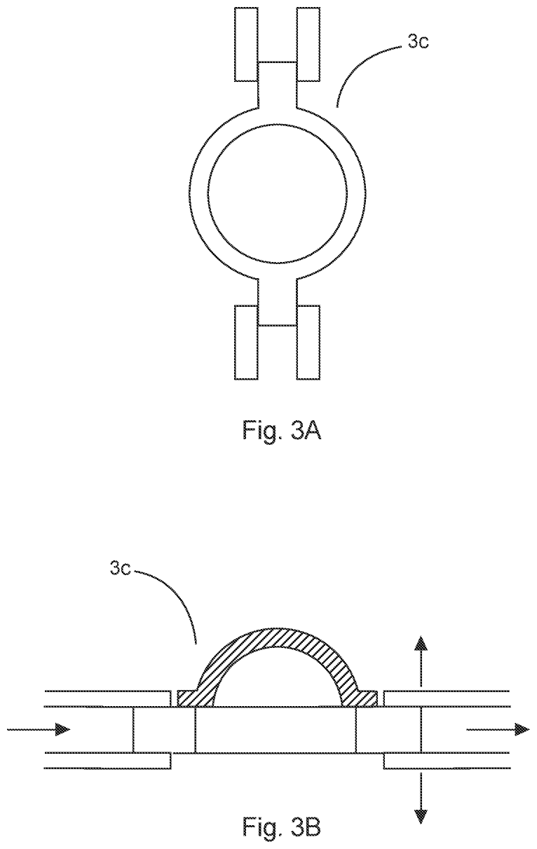

FIG. 3 shows the third embodiment. In this embodiment, the infusion line 2 which is located downstream of the filter element 1 (not shown) and which leads to the patient access port 5 (not shown) can be seen. A line section 3 of the infusion line 2 represents the pressure indicator 3. The pressure indicator 3 is formed here exemplarily as a pressure indicator with an elastic membrane, opposite to which a non-elastic component 3c is located.

1. A medical pressure indicator for infusion, which is combinable with a filter element via an infusion line, wherein the medical pressure indicator is formed as a tube section and is configured to expand and contract in at least one dimension in response to a pressure in the tube section.

2. The medical pressure indicator according to claim 1, wherein the tube section is a line section configured to be arranged downstream of the filter element.

3. The medical pressure indicator according to claim 1, wherein the tube section or at least parts of the tube section of the pressure indicator is or are at least partially formed of elastic or non-elastic material which is shaped to expand and contract.

4. The medical pressure indicator according to claim 1, wherein the tube section expands when the pressure is greater than or equal to 30 mm Hg, and contracts when the pressure is less than or equal to 20 mm Hg.

5. The medical pressure indicator according to claim 1, wherein the tube section is formed as a line section with folds, wherein the tube section is expandable along a longitudinal direction when the pressure is increased and is contractable along the longitudinal direction when the pressure is decreased.

6. The medical pressure indicator according to claim 1, wherein:
the tube section comprises an elastic membrane,
the membrane is expandable in a first direction when the pressure is increased, and
the membrane is expandable in a second direction opposite to the first direction when the pressure is decreased.

7. The medical pressure indicator according to claim 1, wherein the filter element is a capillary membrane.

8. The medical pressure indicator according to claim 1, wherein the tube section expands and contracts by at least 1 mm relative to an unexpanded state.

9. The medical pressure indicator according to claim 1, wherein:
a color marking is applied to the tube section which changes in response to expansion or contraction of the tube section,
or
a color feature on the tube section produces a different color impression depending on a state of elongation.

10. The medical pressure indicator according to claim 1, wherein the tube section has a diameter that is at least 2 mm in a normal state, is at least 3 mm in an expanded state, and is at least 1 mm in a contracted state.

11. A pressure indicator that is sterile, packaged, and configured for connection to an outlet of a medical infusion filter or to a connection of the medical infusion filter, wherein the pressure indicator is adapted to be coupled as a line section for an infusion solution in connection to the outlet of the medical infusion filter or in connection to the connection of the medical infusion filter, wherein the pressure indicator is at least partially made of an elastic material that is expandable and contractable, and wherein the pressure indicator is expandable and contractable in at least one dimension in response to a pressure in the line section.

12. The pressure indicator according to claim 11,
wherein the elastic material expands when the pressure is more than 30 mm Hg and contracts when the pressure is less than 20 mm Hg.

13. The medical pressure indicator according to claim 1, wherein the medical pressure indicator is combined with the filter element.

14. A method of measuring a pressure comprising a step of connecting the pressure indicator according to claim 1 to an outlet of a medical infusion filter.

15. An infusion line comprising a pressure indicator according to claim 11.

16. The infusion line according to claim 15, further comprising an infusion filter.

17. A medical pressure indicator according to claim 2, wherein the line section is a section of an infusion tube or an infusion system.

18. The medical pressure indicator according to claim 4, wherein the tube section expands when the pressure is greater than or equal to 40 mm Hg, and contracts when the pressure is less than or equal to 10 mm Hg.

19. The pressure indicator according to claim 12, wherein the elastic material expands when the pressure is more than 40 mm Hg and contracts when the pressure is less than 10 mm Hg.
